(19) United States
(12) Patent Application Publication
(10) Pub. No.: US 2017/0016069 A1
(43) Pub. Date: Jan. 19, 2017

(54) METHOD FOR TESTING FOR AUTOSOMAL DOMINANT POLYCYSTIC KIDNEY DISEASE AND METHOD FOR SCREENING AGENT FOR TREATMENT OF THE DISEASE

(71) Applicant: KYOTO UNIVERSITY, Kyoto-shi (JP)

(72) Inventors: Kenji Osafune, Kyoto-shi (JP); Tomonaga Ameku, Kyoto-shi (JP); Akira Watanabe, Kyoto-shi (JP)

(73) Assignee: KYOTO UNIVERSITY, Kyoto-shi (JP)

(21) Appl. No.: 15/211,131

(22) Filed: Jul. 15, 2016

(30) Foreign Application Priority Data
Jul. 17, 2015 (JP) 2015-143442

(51) Int. Cl. C12Q 1/68; G01N 33/50

(52) U.S. Cl. CPC C12Q 1/6883; G01N 33/5023; G01N 33/5064; G01N 33/5061; C12Q 2600/158; C12Q 2600/136

(57) ABSTRACT

This invention provides a method of comparing disease markers obtained from subject samples to test for, detect, or diagnose autosomal dominant polycystic kidney disease and a disease marker for such disease. The method for detecting autosomal dominant polycystic kidney disease and the method for screening for an agent for treatment or prevention of such disease comprise detecting a gene that is expressed specifically in cases of autosomal dominant polycystic kidney disease, including IGFBP7.

### TECHNICAL FIELD

The present invention relates to a method and a disease marker for testing for autosomal dominant polycystic kidney disease and a method for screening for an agent for treatment of such disease.

### BACKGROUND ART

In Japan, autosomal dominant polycystic kidney disease (ADPKD) is deduced to occur in one out of approximately 4,000 people, and the number of patients with such disease is deduced to be 20,000 to 50,000. Following diabetic nephropathy, primary glomerulonephritis, and hypertensive nephrosclerosis, ADPKD is the fourth most frequent disease that causes end-stage chronic renal failure leading to the need for dialysis treatments. A major pathological condition of ADPKD in the kidney is the growth of numerous cysts. Examples of pathological conditions found outside the kidney include sacculation in the liver, the pancreas, the spleen, the reproductive organs, and the arachnoid membrane, intracranial and aortic aneurysms, heart valve defects, diverticulum of the large intestine, and hernia. While ADPKD typically occurs during the middle age, a wide range of people from newborn babies to eighty-year-old people are afflicted therewith.

This disease is an autosomal dominant disease caused by a mutation in the PKD1 or PKD2 gene (JP 2001-520502 A, JP 2004-504038 A, and JP 2009-065988 A). However, the sequence encoding such gene is very long, and it is not easy to identify a mutation in such sequence.

Accordingly, development of a method for detecting autosomal dominant polycystic kidney disease at an early stage has been awaited, and a method of diagnosis on the basis of the lowered expression level of the GLIS3 gene has been reported (JP 2006-288265 A). In addition, a gene serving as a marker of autosomal dominant polycystic kidney disease was discovered by sampling cells from a patient with autosomal dominant polycystic kidney disease, establishing iPS cells therefrom, and inducing the iPS cells to develop into vascular endothelial cells or vascular smooth muscle cells (WO 2012/060109).

### SUMMARY OF THE INVENTION

### Objects to be Attained by the Invention

It is an object of the present invention to provide a method of comparing disease markers obtained from subject's samples to test for, detect, or diagnose autosomal dominant polycystic kidney disease and a disease marker for such disease.

It is another object of the present invention to provide, with the use of such disease marker, a method for screening for an agent that is useful for prevention or treatment of autosomal dominant polycystic kidney disease and an agent or medicine that is useful for treatment of such disease.

### Means for Attaining the Objects

The present inventors have conducted concentrated studies in order to attain the above objects. As a result, they discovered that whether or not a subject has developed or is at risk of developing autosomal dominant polycystic kidney disease could be specifically detected using as an indicator an enhanced (increased) expression level of a particular single gene or a plurality of genes or a lowered (decreased) expression level of a particular single gene or a plurality of genes. This has led to the completion of the present invention.

Specifically, the present invention has the following features.

[1] A method for determining whether or not a subject has developed or is at risk of developing autosomal dominant polycystic kidney disease comprising the following steps:

(a) measuring the expression level of a single gene or two to all genes selected from the group consisting of the genes shown in Table 1 or Table 3 in a sample obtained from the subject; and

(b) when the expression level is higher than the expression level of the same gene in a control sample, determining that the subject has developed or is at risk of developing autosomal dominant polycystic kidney disease.

[2] A method for determining whether or not a subject has developed or is at risk of developing autosomal dominant polycystic kidney disease comprising the following steps:

(a) measuring the expression level of a single gene or two to all genes selected from the group consisting of the genes shown in Table 2 or Table 4 in a sample obtained from the subject; and

(b) when the expression level is higher than the expression level of the same gene in a control sample, determining that the subject has not developed or is not at risk of developing autosomal dominant polycystic kidney disease.

[3] The method according to [1] or [2], wherein the sample obtained from the subject is at least one type of sample selected from the group consisting of blood, serum, plasma, cell extract, urine, lymph, tissue fluid, ascites fluid, spinal fluid, another body fluid, a tissue, and a cell.
[4] The method according to [1], wherein the sample obtained from the subject is a vascular endothelial cell induced to differentiate from the iPS cell derived from a somatic cell of the subject and the gene in Step (a) is selected from the group consisting of the genes shown in Table 1.
[5] The method according to [2], wherein the sample obtained from the subject is a vascular endothelial cell induced to differentiate from the iPS cell derived from a somatic cell of the subject and the gene in Step (a) is selected from the group consisting of the genes shown in Table 2.
[6] The method according to [1], wherein the sample obtained from the subject is a vascular smooth muscle cell induced to differentiate from the iPS cell derived from a somatic cell of the subject and the gene in Step (a) is selected from the group consisting of the genes shown in Table 3.
[7] The method according to [2], wherein the sample obtained from the subject is a vascular smooth muscle cell induced to differentiate from the iPS cell derived from a somatic cell of the subject and the gene in Step (a) is selected from the group consisting of the genes shown in Table 4.
[8] A method for screening for an agent for treatment or prevention of autosomal dominant polycystic kidney disease comprising the following steps:

(a) bringing a candidate substance into contact with a vascular endothelial cell induced to differentiate from the iPS cell derived from a somatic cell of a patient with autosomal dominant polycystic kidney disease;

(b) measuring the expression level or transcription activity of a single gene or two to all genes selected from the group consisting of the genes shown in Table 1 and Table 2; and

(c) when the expression level or transcription activity of a single gene or two to all genes selected from the group consisting of the genes shown in Table 1 has decreased in comparison with the case in which the candidate substance has not been brought into contact, determining that the candidate substance is an agent for treatment or prevention of autosomal dominant polycystic kidney disease, or when the expression level or transcription activity of a single gene or two to all genes selected from the group consisting of the genes shown in Table 2 has increased, selecting the candidate substance as an agent for treatment or prevention of autosomal dominant polycystic kidney disease.

[9] A method for screening for an agent for treatment or prevention of autosomal dominant polycystic kidney disease comprising the following steps:

(a) bringing a candidate substance into contact with a vascular smooth muscle cell induced to differentiate from the iPS cell derived from a somatic cell of a patient with autosomal dominant polycystic kidney disease;

(b) measuring the expression level or transcription activity of a single gene or two to all genes selected from the group consisting of the genes shown in Table 3 and Table 4; and

(c) when the expression level or transcription activity of a single gene or two to all genes selected from the group consisting of the genes shown in Table 3 has decreased in comparison with the case in which the candidate substance has not been brought into contact, determining that the candidate substance is an agent for treatment or prevention of autosomal dominant polycystic kidney disease, or when the expression level or transcription activity of a single gene or two to all genes selected from the group consisting of the genes shown in Table 4 has increased, selecting the candidate substance as an agent for treatment or prevention of autosomal dominant polycystic kidney disease.

[10] The screening method according to [8] or [9], wherein the step of measuring the gene expression level comprises measuring the mRNA, cRNA, or cDNA level of the gene.

### Effects of the Invention

According to the method of the present invention, autosomal dominant polycystic kidney disease can be tested for, and an agent that is useful for prevention or treatment of such disease can be screened for.

### EMBODIMENTS FOR CARRYING OUT THE INVENTION

The present invention is based on the finding as described below. That is, whether or not the expression level of at least one gene shown in Table 1 and Table 3 is enhanced (increased) or the expression level of at least one gene shown in Table 2 and Table 4 is lowered (decreased) in comparison with the control sample is determined and the extent of such increase or decrease is qualitatively and/or quantitatively assayed. On the basis thereof, whether or not the subject has developed or is at risk of developing autosomal dominant polycystic kidney disease can be specifically detected, and accurate testing for the disease is thus possible.

Specifically, the present invention provides a disease marker that is useful as a tool enabling the determination of whether or not a subject is afflicted with autosomal dominant polycystic kidney disease and the severity thereof on the basis of the results of qualitative and/or quantitative assays of an increase/decrease in the gene expression level or the extent thereof in the subject. An example of such disease marker is a detection reagent comprising a polynucleotide or antibody.

### <Polynucleotide as Disease Marker>

The present invention provides, as a disease marker of autosomal dominant polycystic kidney disease, a polynucleotide comprising at least 15 continuous nucleotides in an open reading frame (ORF) sequence of any of the nucleotide sequences of the genes shown in Table 1, Table 2, Table 3, and Table 4 and/or a polynucleotide complementary thereto. The ORF sequence of the nucleotide sequence of such gene can be easily obtained on the basis of the NCBI accession number.

The term “complementary polynucleotide (a complementary strand or opposite strand)” used herein refers to a polynucleotide that is complementary to an ORF sequence or a sequence comprising at least 15 continuous nucleotides in the ORF sequence (i.e., a partial sequence) on the basis of a base pair relationship such as A:T and G:C (the ORF sequence and the partial sequence are occasionally referred to as “positive strands” for convenience of description). It should be noted that such complementary strand is not always completely complementary to the nucleotide sequence of the target positive strand and that, with a sufficient degree of complementarity, the complementary strand can hybridize under stringent conditions to the target positive strand. Stringent conditions can be determined on the basis of the melting temperature (Tm) of a nucleic acid connecting a composite or probe as disclosed in Berger and Kimmel (1987, Guide to Molecular Cloning Techniques Methods in Enzymology, Vol. 152, Academic Press, San Diego, Calif.). For example, washing can be generally carried out under conditions such as 1×SSC, 0.1% SDS, and 37° C., following hybridization. A complementary strand is preferably capable of hybridizing to a target positive strand even if it is washed under such conditions. A positive strand can hybridize to a complementary strand even if they are washed under more stringent conditions, such as 0.5×SSC, 0.1% SDS, and 42° C., and even more stringent conditions, such as 0.1×SSC, 0.1% SDS, and 65° C., although the conditions are not necessarily limited thereto. Specific examples of such complementary strands include a strand consisting of a nucleotide sequence that is completely complementary to the nucleotide sequence of the target positive strand and a strand consisting of a nucleotide sequence that has at least 90%, and preferably at least 95%, 96%, 97%, 98%, or 99% sequence identity to such strand.

A polynucleotide in the positive strand can further include a strand consisting of a nucleotide sequence that is complementary to the nucleotide sequence of the complementary strand, in addition to a polynucleotide comprising the ORF sequence or a partial sequence thereof.

In addition, a polynucleotide of the positive strand and a polynucleotide of the complementary strand (opposite strand) may be separately used in the form of a single-stranded as a disease marker, or they may be used in the form of a double-stranded as a disease marker.

As described above, the disease marker of autosomal dominant polycystic kidney disease according to the present invention may be a polynucleotide consisting of an ORF sequence of any of the nucleotide sequences of the genes shown in Table 1, Table 2, Table 3, and Table 4 or a polynucleotide consisting of a sequence complementary thereto. As long as the disease marker can selectively (or specifically) recognize a polynucleotide derived from the gene of interest, it may be a polynucleotide consisting of a partial sequence of the ORF sequence or a sequence complementary thereto. In such a case, a polynucleotide may comprise at least 15, 18, 19, 20, 30, 40, 50, 60, 70, or 100 continuous nucleotides selected arbitrarily from the nucleotide sequence of the ORF sequence or a sequence complementary thereto.

When a polynucleotide derived from the gene of interest can be selectively (or specifically) recognized, the genes shown in Table 1, Table 2, Table 3, and Table 4 or polynucleotides derived therefrom can be specifically detected via, for example, Northern blotting or microarray techniques. When RT-PCR is carried out, the genes shown in Table 1, Table 2, Table 3, and Table 4 or polynucleotides derived therefrom are specifically amplified and generated. The conditions are not limited thereto, and it is sufficient if a person skilled in the art is capable of determining that the product detected via Northern blotting or microarray techniques or a product of RT-PCR is derived from any of the genes shown in Table 1, Table 2, Table 3, and Table 4 or polynucleotides derived therefrom.

Such disease marker according to the present invention can be designed on the basis of the nucleotide sequence of the gene of interest with the use of, for example, Primer 3 (http://primer3.ut.ee/) or Vector NTI (Infomax).

Specifically, a candidate sequence of a primer or probe that is obtained via application of any of the nucleotide sequences of the genes shown in Table 1, Table 2, Table 3, and Table 4 to software such as Primer 3 or Vector NTI, or a sequence comprising such a sequence in part, can be used as a primer or probe.

The disease marker used in the present invention comprises at least 15, 18, 19, 20, 30, 40, 50, 60, 70, or 100 continuous nucleotides as described above. Specifically, the length of the sequence can be adequately determined in accordance with the application of the marker.

The disease marker according to the present invention can be used as a primer that specifically recognizes and amplifies an RNA generated upon expression/transcription of the gene or a polynucleotide derived therefrom (e.g., cDNA), or it can be used as a probe that specifically detects such RNA or a polynucleotide derived therefrom (e.g., cDNA).

When the disease marker is used as a primer for testing for or detecting autosomal dominant polycystic kidney disease, for example, the nucleotide length thereof can be generally 15 bp to 100 bp, preferably 15 bp to 50 bp, and more preferably 20 bp to 35 bp. When the disease marker is used as a detection probe, for example, the nucleotide length thereof can be generally 15 bp to all nucleotides, preferably 15 bp to 1 kb, and more preferably 50 bp to 500 bp.

When the disease marker according to the present invention is used as a probe, the probe may be labeled with a radioactive isotope (e.g., ³²P or ³³P), a fluorescent substance (e.g., fluorescamine, rhodamine, Texas Red, dansyl, or a derivative thereof), a chemoluminescent substance, or an enzyme. Such labeled disease marker can be preferably used as a probe (i.e., a detection marker).

The disease marker according to the present invention can be used as a primer or probe in accordance with a conventional technique known in the art comprising specifically recognizing a particular gene, mRNA, or cDNA and detecting the same, such as Northern blotting, microarray techniques, Southern blotting, RT-PCR, or in situ hybridization.

### <Antibody as Disease Marker>

The present invention also provides, as a disease marker of autosomal dominant polycystic kidney disease, an antibody that can specifically recognize expression products (proteins) of the genes shown in Table 1, Table 2, Table 3, and Table 4.

The form of the antibody according to the present invention is not particularly limited, and such antibody may be a polyclonal antibody or a monoclonal antibody that can recognize any of the proteins shown in Table 1, Table 2, Table 3, and Table 4 or a part thereof as an immunogen. The antibody may be a chimeric antibody such as a human/mouse chimeric antibody, a humanized antibody, a human antibody, or a fragment of any of such antibody (e.g., Fab, Fab′, F(ab′)₂, Fc, Fv, or scFv). A part of a protein may be a polypeptide consisting of at least 8 continuous amino acids, such as 10 to 20 amino acids, in the amino acid sequence of the protein.

Techniques for antibody production are well known in the art, and the antibody according to the present invention can be produced in accordance with such conventional techniques (Current protocols in Molecular Biology, Ausubel et al. (edited), 1987, John Wiley and Sons (published), Section 11.12-11.13).

When the antibody according to the present invention is a polyclonal antibody, specifically, proteins encoded by the genes shown in Table 1, Table 2, Table 3, and Table 4 may be expressed in E. coli or the like and purified in accordance with conventional techniques or oligopeptides comprising partial amino acid sequences may be synthesized, nonhuman animals such as rabbits may be immunized therewith, and the antibody of interest can be obtained from the sera of the immunized animals in accordance with conventional techniques. Nonhuman animals may be immunized by enhancing immunological responses with the use of various adjuvants in accordance with host animal species. Examples of such adjuvants include, but are not limited to, Freund's adjuvants, mineral gels such as aluminum hydroxide, surface active substances such as lysolecithin, Pluronic polyol, polyanion, peptide, oil emulsion, keyhole limpet hemocyanin, and dinitrophenol, and human adjuvants such as BCG (Bacillus Calmette-Guerin) and Corynebacterium parvum.

In the case of the monoclonal antibody, in contrast, the spleen cells and the myeloma cells obtained from the immunized nonhuman animals are fused to each other so as to prepare hybridoma cells, and the antibody can be obtained from the prepared hybridoma cells via, for example, HAT selection and affinity assays with the target polypeptide (Current protocols in Molecular Biology, Ausubel et al. (edited), 1987, John Wiley and Sons (published), Section 11.4-11.11).

Proteins used for antibody production can be obtained, on the basis of sequence information on the genes shown in Table 1, Table 2, Table 3, and Table 4 via DNA cloning, construction of plasmids, transfection into hosts, culture of transformants, and recovery of proteins from culture products. Such procedures can be carried out in accordance with, for example, methods known to a person skilled in the art and methods described in literature (Molecular Cloning, T. Maniatis et al., CSH Laboratory, 1983, DNA Cloning, DM. Glover, IRL PRESS, 1985). Specifically, recombinant DNA enabling a gene to be expressed in a host cell of interest may be prepared (i.e., an expression vector), the recombinant DNA may be introduced into the host cell, the resulting transformant may be cultured, and the target protein may then be recovered from the culture product. Alternatively, proteins can be produced via general chemical synthesis (peptide synthesis) techniques in accordance with the information on the amino acid sequences encoded by the genes shown in Table 1, Table 2, Table 3, and Table 4.

The proteins encoded by the genes shown in Table 1, Table 2, Table 3, and Table 4 according to the present invention encompass homologs thereof. For example, such a homolog may be a protein consisting of an amino acid sequence having 1 or more, and preferably 1 or several amino acid deletion, substitution, or addition in the amino acid sequence encoded by the gene of interest or a protein consisting of an amino acid sequence having at least 90%, preferably at least 95%, 96%, or 97%, further preferably at least 98%, and most preferably at least 99% sequence identity to the amino acid sequence encoded by the gene of interest, which has equivalent biological functions and/or equivalent immunological activity. A mutant resulting from a mutation such as racial polymorphism, mutation, or splice mutation is within the scope of such homolog.

The term “sequence identity” used herein is defined in percentage (%) terms, and refers to the number of the identical amino acid residues or nucleotides relative to the total number of amino acid residues or nucleotides when two amino acid sequences or two nucleotide sequences are aligned with or without the introduction of gaps so as to maximize the degree of amino acid or nucleotide identity. Sequence identity can be determined with the use of, for example, BLAST, which can be found on the NCBI server (i.e., ncbi.nlm.nih.gov/BLAST/) (Altschul S F, et al., 1997, Nucleic Acids Res. 25 (17): 3389-402 or 1990, J. Mol. Biol., 215 (3): 403-10).

The number of amino acid mutations or the sites of mutations in a protein are not limited, provided that the relevant biological functions and/or immunological activity are retained. Indicators to be employed for determination of the manner and the number of amino acid residues to be substituted, inserted, or deleted without loss of the biological functions and/or immunological activity can be found with the use of a computer program well known in the art, such as DNA Star software. For example, the number of mutations is typically within 10%, preferably within 5%, and more preferably within 1% of the total number of amino acids. Amino acids to be substituted are not particularly limited, provided that a protein resulting from substitution of such amino acids retains equivalent levels of biological functions and/or immunological activity. From the viewpoint of retention of protein structure, amino acids preferably have electrical, structural, and other properties similar to those of amino acids before substitution in terms of, for example, polarity, electric charge, solubility, hydrophobic properties, hydrophilic properties, or amphipathic properties of residues. For example, Ala, Val, Leu, Ile, Pro, Met, Phe, and Trp are classified as nonpolar amino acids, Gly, Ser, Thr, Cys, Tyr, Asn, and Gln are classified as uncharged amino acids, Asp and Glu are classified as acidic amino acids, and Lys, Arg, and His are classified as basic amino acids. Thus, adequate amino acids can be selected from among the amino acids of the same group using such amino acid properties as the indicators.

The antibody of the present invention reacting with the protein encoded by any of the genes shown in Table 1, Table 2, Table 3, and Table 4 is capable of specifically binding to the protein encoded by any of the genes shown in Table 1, Table 2, Table 3, and Table 4. With the use of such antibody, accordingly, the protein of interest contained in the sample obtained from the subject can be specifically detected and quantified. Specifically, the antibody of the present invention is useful for testing for, detecting, or diagnosing autosomal dominant polycystic kidney disease.

### <Method for Testing for Autosomal Dominant Polycystic Kidney Disease>

The present invention provides a method for testing for autosomal dominant polycystic kidney disease comprising the following steps (a-1) and (b-1):

(a-1) measuring the expression level of a single gene or two to all genes selected from the group consisting of the genes shown in Table 1 or Table 3 in a sample obtained from the subject; and

(b-1) when the expression level is higher than the expression level of the same gene in a control sample, determining that the subject has developed or is at risk of developing autosomal dominant polycystic kidney disease.

The present invention also provides a method for testing for autosomal dominant polycystic kidney disease comprising the following steps (a-2) and (b-2):

(a-2) measuring the expression level of a single gene or two to all genes selected from the group consisting of the genes shown in Table 2 or Table 4 in a sample obtained from the subject; and

(b-2) when the expression level is higher than the expression level of the same gene in a control sample, determining that the subject has not developed or is not at risk of developing autosomal dominant polycystic kidney disease.

The term “control sample” used herein preferably refers to a sample obtained from a healthy volunteer who is not afflicted with autosomal dominant polycystic kidney disease, unless otherwise specified. In the present invention, the term “healthy volunteer” refers to an individual who is at least not afflicted with autosomal dominant polycystic kidney disease. Whether or not a healthy volunteer is afflicted with other diseases or infections is not a significant issue of concern. A sample obtained from a healthy volunteer can be prepared in the same manner as in the case of the sample derived from the subject. Also, the term “expression level in a control sample” refers to the results of measurement of the expression level of a given gene obtained from the subject in a similar manner.

When the expression level is “high” in the present invention, for example, such expression level is higher than the level in the control sample. When the expression level is at least 1.5 times, 2 times, 3 times, preferably 5 times, and more preferably 10 times higher than the level in the control sample, for example, whether or not the subject has developed or is at risk of developing the disease can be determined with higher reliability.

In the present invention, blood, serum, plasma, cell extract, urine, lymph, tissue fluid, ascites fluid, spinal fluid, another body fluid, tissue, or cell (e.g., renal tissue, renal cell, or somatic cell induced to differentiate from iPS cell) samples obtained from the subject or a healthy volunteer can be used. Examples of somatic cells induced to differentiate from iPS cells include tubular cells, collecting tubule cells, bile duct cells, hepatic cells, pancreatic ductal cells, pancreatic cells, intestinal cells, germ cells, vascular endothelial cells, and vascular smooth muscle cells. Methods for producing tubular cells, collecting tubule cells, bile duct cells, hepatic cells, pancreatic ductal cells, pancreatic cells, intestinal cells, germ cells, vascular endothelial cells, or vascular smooth muscle cells from iPS cells are not particularly limited. These cells can be adequately extracted from the embryoid body or the developed teratoma (e.g., JP 2006-239169 A). Hepatic cells can be produced by the methods disclosed in WO 2006/082890, JP 2010-75631 A, or Hay D C, et al., Proc. Natl. Acad. Sci., U.S.A., 105, 12301-6, 2008, although the methods are not particularly limited thereto. Also, pancreatic cells can be produced by the method disclosed in WO 2007/103282. In addition, iPS cells, vascular endothelial cells, or vascular smooth muscle cells can be produced by the method described below.

When vascular endothelial cells induced to differentiate from iPS cells derived from somatic cells of the subject are used as the sample obtained from the subject, the present invention provides the method for testing for autosomal dominant polycystic kidney disease comprising the following steps (a-3) and (b-3):

(a-3) measuring the expression level of a single gene or two to all genes selected from the group consisting of the genes shown in Table 1 in the vascular endothelial cells induced to differentiate from iPS cells derived from somatic cells of the subject; and

(b-3) when the expression level is higher than the expression level of the same gene in a control sample, determining that the subject has developed or is at risk of developing autosomal dominant polycystic kidney disease.

The present invention also provides the method for testing for autosomal dominant polycystic kidney disease comprising the following steps (a-4) and (b-4):

(a-4) measuring the expression level of a single gene or two to all genes selected from the group consisting of the genes shown in Table 2 in the vascular endothelial cells induced to differentiate from iPS cells derived from somatic cells of the subject; and

(b-4) when the expression level is higher than the expression level of the same gene in a control sample, determining that the subject has not developed or is not at risk of developing autosomal dominant polycystic kidney disease.

Another embodiment of the present invention provides the method for testing for autosomal dominant polycystic kidney disease comprising the following steps (a-5) and (b-5) when the vascular smooth muscle cells induced to differentiate from iPS cells derived from somatic cells of the subject are used as the sample obtained from the subject:

(a-5) measuring the expression level of a single gene or two to all genes selected from the group consisting of the genes shown in Table 3 in the vascular smooth muscle cells induced to differentiate from iPS cells derived from somatic cells of the subject; and

(b-5) when the expression level is higher than the expression level of the same gene in a control sample, determining that the subject has developed or is at risk of developing autosomal dominant polycystic kidney disease.

The present invention further provides the method for testing for autosomal dominant polycystic kidney disease comprising the following steps (a-6) and (b-6):

(a-6) measuring the expression level of a single gene or two to all genes selected from the group consisting of the genes shown in Table 4 in the vascular smooth muscle cells induced to differentiate from iPS cells derived from somatic cells of the subject; and

(b-6) when the expression level is higher than the expression level of the same gene in a control sample, determining that the subject has not developed or is not at risk of developing autosomal dominant polycystic kidney disease.

In the present invention, the gene expression level can be measured with the use of the disease marker comprising the polynucleotide or antibody described above.

When a polynucleotide is used as a disease marker, a sample is preferably cells isolated from the subject or somatic cells induced to differentiate from iPS cells.

When mRNA, non-coding RNA, or a polynucleotide prepared therefrom (e.g., cDNA or cRNA) is used as an analyte, the following step can be performed:

(i) a step of binding mRNA prepared from the subject's sample, non-coding RNA, or a complementary polynucleotide transcribed therefrom to the disease marker; and

(ii) a step of measuring the amount of RNA derived from the subject's sample bound to the disease marker or a complementary polynucleotide (cDNA) transcribed from the RNA using the abundance of the disease marker as the indicator.

In Step (ii), measurement can be carried out with the use of the disease marker consisting of the polynucleotide described above as a primer or probe by subjecting the mRNA or the like to conventional techniques, such as Northern blotting, Southern blotting, RT-PCR, microarray techniques, or in situ hybridization analysis.

When Northern blotting or Southern blotting is employed, the disease marker of the present invention may be used as a probe, so that the expression level of the target gene in mRNA or the like can be determined or measured. Specifically, the disease marker of the present invention (a complementary strand for RNA) may be labeled with, for example, a radioactive isotope (RI, such as ³²P or ³³P) or a fluorescent substance, the resultant may be allowed to hybridize to mRNA derived from a biological tissue sample of the subject transferred to a nylon membrane or the like in accordance with a conventional technique, and the resulting double strand of the disease marker and mRNA or the like may be detected or measured on the basis of a signal derived from the labeled disease marker (e.g., RI or a fluorescent substance) using a radiation detector (Typhoon FLA 9000, GE Healthcare) or a fluorescence detector. Alternatively, the AlkPhos Direct Labeling and Detection System (Amersham Pharmacia Biotech) may be used, the disease marker may be labeled in accordance with the instruction of the system, the labeled product may then be allowed to hybridize to mRNA or the like derived from the biological tissue sample of the subject, and a signal derived from the labeled disease marker may be detected or measured with the use of the Multi Bio Imager (STORM 860, Amersham Pharmacia Biotech).

When RT-PCR is performed, the disease marker of the present invention is used as a primer, so that the gene expression level in RNA or the like can be detected or measured. Specifically, cDNA is prepared from RNA obtained from the subject's sample in accordance with a conventional technique, the disease marker of the present invention is used as a primer so as to amplify the target gene region with the use of the prepared cDNA as a template, PCR is performed in accordance with a conventional technique, and the resulting amplified double-stranded DNA can be detected.

PCR is carried out by repeating a cycle of denaturation, annealing, and extension, for example, 20 to 40 times. A process of denaturation is carried out to divide double-stranded DNA into single-stranded DNAs, and this process is generally carried out at 94° C. to 98° C. for about 10 seconds to 2 minutes. A process of annealing is carried out to bind a sense primer or an antisense primer to single-stranded template DNA, and this process is generally carried out at 50° C. to 68° C. for about 10 seconds to 1 minute. A process of extension is carried out to extend a primer along template DNA, and this process is generally carried out at 72° C. for about 20 seconds to 10 minutes. Before the above-described cycle is initiated, double-stranded DNA may be pre-treated under the same conditions as denaturation conditions. After the completion of the above-described cycle, post-treatment may be carried out under the same conditions as extension conditions. PCR involves the use of a PCR buffer and a thermostable DNA polymerase, and the amplified product can be examined via, for example, electrophoresis. PCR can be carried out with the use of a commercially available PCR apparatus, such as a thermal cycler.

When microarrays are used, further, a DNA chip to which the disease marker of the present invention is applied as a DNA probe (a single-stranded or double-stranded polynucleotide) is prepared, the DNA chip is subjected to hybridization with cRNA prepared from RNA obtained from the biological tissue of the subject in accordance with a conventional technique, and the disease marker of the present invention labeled with RI, a fluorescent substance, or the like is allowed to bind to the double strand of DNA and cRNA as a label probe, so as to detect the gene of interest. An example of a DNA chip capable of detection or measurement of gene expression levels is the Gene Chip (Affymetrix).

When a protein is an analyte, the protein is brought into contact with the antibody that is the disease marker of the present invention and the protein or a partial peptide thereof bound to the antibody is detected by a known detection method, such as Western blotting or enzyme-linked immunosorbent assays (ELISA), using the disease marker of the present invention as the indicator and quantified.

Western blotting can be carried out in the manner described below. That is, the antibody that is the disease marker of the present invention is used as a primary antibody, an antibody labeled with a radioactive isotope such as ¹²⁵I, an enzyme such as horseradish peroxidase (HRP), or a fluorescent substance capable of binding to the primary antibody is used as a secondary antibody, and a composite of a protein or a partial peptide thereof and the disease marker (i.e., the primary antibody) is labeled. Subsequently, a signal derived from the radioactive isotope or fluorescent substance is detected or measured using a radiation detector (Typhoon FLA 9000, GE Healthcare) or a fluorescence detector. Alternatively, the antibody that is the disease marker of the present invention may be used as a primary antibody, detection may be carried out using the ECL Plus Western Blotting Detection System (Amersham Pharmacia Biotech) in accordance with the instructions for use of the system, and measurement may then be carried out with the use of the Multi Bio Imager (STORM 860, Amersham Pharmacia Biotech).

ELISA (e.g., sandwich ELISA) can be carried in accordance with a method known to a person skilled in the art. Specifically, a solution containing the antibody that is the disease marker of the present invention is added and fixed to a support, such as a plate, as a primary antibody. The plate is washed and then blocked with, for example, BSA, so as to prevent nonspecific protein binding. The plate is washed again, and the sample is then applied to the plate. Following incubation, the plate is washed, and a labeled antibody such as a biotin-labeled antibody is added as a secondary antibody. After incubation has been adequately carried out, the plate is washed, and avidin bound to an enzyme, such as alkaline phosphatase or peroxidase, is added thereto. Following incubation, the plate is washed, a substrate is added thereto in accordance with a type of an enzyme bound to avidin, and the protein level of interest is detected using the enzymatic change of the substrate as an indicator.

### <Method for iPS Cell Production>

Induced pluripotent stem (iPS) cells can be prepared by allowing a particular reprogramming factor to react with somatic cells. iPS cells are artificial stem cells derived from somatic cells having properties that are substantially equivalent to those of ES cells (K. Takahashi and S. Yamanaka, 2006, Cell, 126: 663-676; K. Takahashi et al., 2007, Cell, 131: 861-872; J. Yu et al., 2007, Science, 318: 1917-1920; Nakagawa, M. et al., Nat. Biotechnol. 26: 101-106, 2008; WO 2007/069666).

A reprogramming factor may be composed of a gene that is expressed specifically in ES cells, a gene product or non-coding RNA thereof, a gene that plays a key role in maintaining ES cells in an undifferentiated state, a gene product or non-coding RNA thereof, or a low-molecular-weight compound. Examples of genes contained in the reprogramming factor include Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tcl1, beta-catenin, Lin28b, Sall1, Sall4, Esrrb, Nr5a2, Tbx3, and Glis1. A single type of such reprogramming factor may be used alone, or two or more types of such reprogramming factors may be used in combination. Reprogramming factors can be used in known combination and examples of such combinations are described in WO 2007/069666, WO 2008/118820, WO 2009/007852, WO 2009/032194, WO 2009/058413, WO 2009/057831, WO 2009/075119, WO 2009/079007, WO 2009/091659, WO 2009/101084, WO 2009/101407, WO 2009/102983, WO 2009/114949, WO 2009/117439, WO 2009/126250, WO 2009/126251, WO 2009/126655, WO 2009/157593, WO 2010/009015, WO 2010/033906, WO 2010/033920, WO 2010/042800, WO 2010/050626, WO 2010/056831, WO 2010/068955, WO 2010/098419, WO 2010/102267, WO 2010/111409, WO 2010/111422, WO 2010/115050, WO 2010/124290, WO 2010/147395, WO 2010/147612, Huangfu D, et al., 2008, Nat. Biotechnol., 26: 795-797, Shi Y, et al., 2008, Cell Stem Cell, 2: 525-528, Eminli S, et al., 2008, Stem Cells. 26: 2467-2474, Huangfu D, et al., 2008, Nat. Biotechnol. 26: 1269-1275, Shi Y, et al., 2008, Cell Stem Cell, 3, 568-574, Zhao Y, et al., 2008, Cell Stem Cell, 3: 475-479, Marson A, 2008, Cell Stem Cell, 3, 132-135, Feng B, et al., 2009, Nat. Cell Biol., 11: 197-203, R. L. Judson et al., 2009, Nat. Biotechnol., 27: 459-461, Lyssiotis C A, et al., 2009, Proc. Natl. Acad. Sci. U.S.A., 106: 8912-8917, Kim J B, et al., 2009, Nature, 461: 649-643, Ichida J K, et al., 2009, Cell Stem Cell. 5: 491-503, Heng J C, et al., 2010, Cell Stem Cell, 6: 167-74, Han J., et al., 2010, Nature, 463: 1096-100, Mali P, et al., 2010, Stem Cells, 28: 713-720, and Maekawa, M., et al., 2011, Nature, 474: 225-9.

A reprogramming factor may be brought into contact with somatic cells or introduced into somatic cells by a conventional technique in accordance with its form.

When a reprogramming factor is in the form of a protein, it may be introduced into somatic cells via, for example, lipofection, fusion to a cell-permeable peptide (e.g., HIV-derived TAT and polyarginine), or microinjection.

When a reprogramming factor is in the form of DNA, for example, it may be introduced into somatic cells with the use of a vector, such as a virus, plasmid, or artificial chromosome vector or via a technique such as lipofection, liposome, or microinjection. Examples of virus vectors include retrovirus vector, lentivirus vector (Cell, 126, pp. 663-676, 2006; Cell, 131, pp. 861-872, 2007; Science, 318, pp. 1917-1920, 2007), adenovirus vector (Science, 322, 945-949, 2008), adeno-associated virus vector, and Sendai virus vector (WO 2010/008054). Examples of artificial chromosome vectors include human artificial chromosome (HAC), yeast artificial chromosome (YAC), and bacterial artificial chromosome (BAC or PAC). As a plasmid, a plasmid for mammalian animal cells can be used (Science, 322: 949-953, 2008). A vector can comprise a regulatory sequence, such as a promoter, an enhancer, a ribosome binding sequence, a terminator, or a polyadenylation site, so that a nuclear reprogramming substance can be expressed. In addition, a vector can comprise a selection marker sequence, such as a drug-tolerant gene (e.g., the kanamycin tolerant gene, the ampicillin tolerant gene, or the puromycin tolerant gene), the thymidine kinase gene, or the diphtheria toxin gene, and a reporter gene sequence, such as a green fluorescent protein (GFP), β glucuronidase (GUS), or FLAG, according to need. The vector may be first introduced into and allowed to react with somatic cells, and a gene encoding a reprogramming factor or a promoter and a gene encoding a reprogramming factor binding thereto may be cleaved together. To this end, the gene encoding a reprogramming factor or the promoter and the gene encoding a reprogramming factor binding thereto may be flanked by LoxP sequences.

When a reprogramming factor is in the form of RNA, the reprogramming factor may be introduced into somatic cells via, for example, lipofection or microinjection. In order to suppress decomposition, RNA into which 5-methylcytidine and pseudouridine (TriLink Biotechnologies) have been incorporated may be used as a reprogramming factor (Warren L., 2010, Cell Stem Cell, 7: 618-630).

Examples of culture media used for iPS cell induction include DMEM, DMEM/F12, and DME containing 10% to 15% FBS. These culture media can further contain LIF, penicillin/streptomycin, puromycin, L-glutamine, non-essential amino acids, or β-mercaptoethanol, according to need. Other examples include commercially available culture media (e.g., a mouse ES cell culture medium; TX-WES medium, Thromb-X), a primate ES cell culture medium (e.g., a primate ES/iPS cell culture medium, ReproCELL Inc.), and a serum-free medium (mTeSR, Stemcell Technology).

iPS cells can be induced in the manner described below. For example, somatic cells are brought into contact with reprogramming factors at 37° C. in the presence of 5% CO₂ in a DMEM or DMEM/F12 medium containing 10% FBS, culture is conducted for approximately 4 to 7 days, the cells are reseeded on feeder cells (e.g., mitomycin C-treated STO cells or SNL cells), and culture is restarted in a bFGF-containing primate ES cell culture medium about 10 days after the somatic cells have been brought into contact with the reprogramming factors. Thus, ES-like colonies can be formed about 30 to 45 days or more after contact.

Alternatively, somatic cells are brought into contact with reprogramming factors at 37° C. in the presence of 5% CO₂ in a 10% FBS-containing DMEM medium (this medium can further contain LIF, penicillin/streptomycin, puromycin, L-glutamine, non-essential amino acids, or β-mercaptoethanol, according to need) on feeder cells (e.g., mitomycin C-treated STO cells or SNL cells), culture is conducted, and ES-like colonies can be formed about 25 to 30 days or more thereafter. Instead of feeder cells, preferably, the somatic cells to be reprogrammed (Takahashi, K., et al., 2009, PLoS One, 4: e8067 or WO 2010/137746) or extracellular matrices (e.g., Laminin-5 (WO 2009/123349), Laminin-10 (US 2008/0213885), a fragment thereof (WO 2011/043405), or Matrigel (BD)) are used.

Alternatively, iPS cells can be established with the use of a serum-free medium (Sun, N., et al., 2009, Proc. Natl. Acad. Sci., U.S.A., 106: 15720-15725). In order to further enhance establishment efficiency, iPS cells may be established under reduced oxygen conditions (oxygen concentration: 0.1% or more and 15% or less) (Yoshida, Y., et al., 2009, Cell Stem Cell, 5: 237-241 or WO 2010/013845).

Examples of components that are known to enhance iPS cell establishment efficiency include histone deacetylase (HDAC) inhibitors (e.g., low-molecular-weight inhibitors, such as valproic acid (VPA), trichostatin A, sodium butyrate, MC 1293, and M344, and nucleic acid-based expression inhibitors, such as siRNA and shRNA against HDAC (e.g., HDAC1 siRNA Smartpool® (Millipore) and HuSH 29mer shRNA constructs against HDAC1 (OriGene)), MEK inhibitors (e.g., PD184352, PD98059, U0126, SL327, and PD0325901), glycogen synthase kinase-3 inhibitors (e.g., Bio and CHIR99021), DNA methyl transferase inhibitors (e.g., 5-azacytidine), histone methyl transferase inhibitors (e.g., low-molecular-weight inhibitors, such as BIX-01294, and nucleic acid-based expression inhibitors, such as siRNA and shRNA against Suv39h1, Suv39h2, SetDB1, and G9a), L-channel calcium agonists (e.g., Bayk8644), butyric acid, TGFβ inhibitors or ALK5 inhibitors (e.g., LY364947, SB431542, 616453, and A-83-01), p53 inhibitors (e.g., siRNA and shRNA against p53), ARID3A inhibitors (e.g., siRNA and shRNA against ARID3A), miRNAs, such as miR-291-3p, miR-294, miR-295, and mir-302, Wnt Signaling (e.g., soluble Wnt3a), neuro-peptide Y, prostaglandins (e.g., prostaglandin E2 and prostaglandin J2), hTERT, SV40LT, UTF1, IRX6, GLIS1, PITX2, and DMRTB1. When establishing iPS cells, a culture medium supplemented with such components aimed at improvement of the establishment efficiency may be used.

During the culture, a culture medium is exchanged with a fresh medium once every day, and such exchange is initiated 2 days after the initiation of culture. The number of somatic cells used for nuclear reprogramming is not limited, and the number of cells is about 5×10³ to 5×10⁶ cells/100 cm² of the culture dish.

iPS cells can be selected in accordance with the forms of the developed colonies. Alternatively, a drug-tolerant gene expressed in conjunction with the gene (e.g., Oct3/4, Nanog) expressed when somatic cells are reprogrammed is introduced as a marker gene, culture is conducted in a culture medium containing an appropriate agent (a selection medium), and the established iPS cells can be selected. Also, a fluorescent protein gene may be introduced as a marker gene and observed under a fluorescent microscope whereby iPS cells can be selected. In the case of a luciferase gene, iPS cells can be selected with the addition of a luminescent substrate.

Examples of “somatic cells” used for iPS cell induction used herein include, but are not limited to, keratinizing epithelial cells (e.g., keratinizing epidermal cells), mucosal epithelial cells (e.g., epithelial cells of the surface layer of tongue), exocrine epithelial cells (e.g., mammary glandular cells), hormone-secreting cells (e.g., adrenal medullary cells), cells for metabolism/storage (e.g., hepatic cells), boundary-forming luminal epithelial cells (e.g., type I alveolar cells), luminal epithelial cells of internal tubules (e.g., vascular endothelial cells), ciliated cells having transport capacity (e.g., tracheal epithelial cells), cells for extracellular matrix secretion (e.g., fibroblasts), contractile cells (e.g., smooth muscle cells), cells of the blood and the immune system (e.g., T lymphocytes), sense-related cells (e.g., rod cells), autonomic neurons (e.g., cholinergic neurons), sustentacular cells of sensory organs and periphery neurons (e.g., satellite cells), neurons and glia cells in the central nervous system (e.g., astroglia cells), pigment cells (e.g., retinal pigment epithelial cells), and progenitor cells (tissue progenitor cells) thereof. Somatic cells are not particularly limited in terms of the extent of cell differentiation. Undifferentiated progenitor cells (including somatic stem cells) and mature cells after the completion of the final differentiation can also be used as the origins of the somatic cells in the present invention. Examples of undifferentiated progenitor cells include tissue stem cells (somatic stem cells), such as neural stem cells, hematopoietic stem cells, mesenchymal stem cells, and dental pulp stem cells.

<Method for Inducing Differentiation into Vascular Endothelial Cells>

Vascular endothelial cells can be produced from the iPS cells obtained in the manner described above by the method of differentiation induction comprising the following steps:

(1) performing adhesion culture using a primate ES/iPS cell culture medium on a coated culture dish;

(2) performing culture with the addition of various additives to the medium;

(3) performing culture with the addition of growth factors to a serum-free medium;

(4) separating VEGFR2-positive, TRA1-negative, and VE-cadherin-positive cells; and

(5) performing adhesion culture using a vascular endothelial cell growth medium on a coated culture dish.

According to the present invention, preferably, the vascular endothelial cells express vascular endothelial cell markers, such as VE-cadherin, CD31, CD34, and eNOS, and such cells have cobblestone appearances.

iPS cells can be detached by any method prior to Step (1). iPS cells may be detached with the use of a mechanical process, a detachment solution having protease activity and collagenase activity (e.g., Accutase™ or Accumax™) or a separation liquid having collagenase activity only.

Examples of coating agents used in Step (1) and Step (5) include Matrigel (BD), type I collagen, type IV collagen, gelatin, laminin, heparan sulfate proteoglycan, entactin, and a combination of any thereof. Type I collagen is preferably used in Step (1) and type IV collagen is preferably used in Step (5).

A medium used for preparing vascular endothelial cells can be prepared using a medium for animal cell culture as a basal medium. Examples of basal medium include IMDM medium, Medium 199, Eagle's Minimum Essential Medium (EMEM), aMEM medium, Dulbecco's modified Eagle's Medium (DMEM), Ham's F12 medium, RPMI 1640 medium, Fischer's medium, and a mixture of any thereof. A medium may further contain serum, or it may be a serum-free medium. According to need, a medium can contain, for example, one or more serum alternatives selected from among, for example, albumin, transferrin, knockout serum replacement (KSR) (a serum alternative for FBS when ES cells are cultured), fatty acid, insulin, collagen precursor, trace elements, 2-mercaptoethanol, and 3′-thiol glycerol. A medium can contain one or more substances selected from among lipids, amino acid, L-glutamine, Glutamax (Invitrogen), non-essential amino acids, vitamins, antibiotics, antioxidants, pyruvic acid, buffers, inorganic salts, N2 supplement (Invitrogen), B27 supplement (Invitrogen), GSK-3α/β inhibitor, and a growth factor such as VEGF. Examples of media supplemented with such additives include primate ES/iPS cell culture medium (ReproCELL), Stem Pro™ (Invitrogen), and vascular endothelial cell growth medium (Lonza). Examples of preferable media used in the present invention are: a primate ES/iPS cell culture medium used in Step (1); a primate ES/iPS cell culture medium supplemented with N2 supplement, B27 supplement, and a GSK-3α/β inhibitor used in Step (2); VEGF-containing Stem Pro™ used in Step (3); and vascular endothelial cell growth medium used in Step (5).

Examples of GSK-3α/β inhibitors include SB216763, SB415286, FRAT1/FRAT2, Lithium, Kempaullone, Alsterpaullone, Indiubin-3′-oxime, BIO, TDZD-8, and Ro31-8220.

Culture temperature is about 30° C. to 40° C., and preferably about 37° C., although it is not limited thereto. Culture is conducted in atmosphere containing CO₂, and the preferable CO₂ concentration is about 2% to 5%. While the culture duration is not particularly limited, for example, Step (1) is preferably performed for 1 to 2 days, and more preferably for 1 day, Step (2) is preferably performed for 2 to 5 days, and more preferably for 3 days, Step (3) is preferably performed for 3 to 7 days, and more preferably for 5 days, and Step (5) is preferably performed for at least 3 days.

VEGFR2-positive, TRA1-negative, and VE-cadherin-positive cells can be separated from the cells stained with antibodies reacting with VEGFR2, TRA1, and VE-cadherin with the use of a flow cytometer or other means in accordance with a method well known to a person skilled in the art.

<Method for Inducing Differentiation into Vascular Smooth Muscle Cells>

Vascular smooth muscle cells can be produced by the method of differentiation induction comprising the same steps as Steps (1) to (3) used in the method for producing vascular endothelial cells described above and subsequent Steps (4′) and (5′) described below:

(1) performing adhesion culture using a primate ES/iPS cell culture medium on a coated culture dish;

(2) performing culture with the addition of various additives to the medium;

(3) performing culture with the addition of growth factors to a serum-free medium;

(4′) separating VEGFR2-positive, TRA1-negative, and VE-cadherin-negative cells; and

(5′) performing adhesion culture using a growth factor-containing medium on a coated culture dish.

In the present invention, preferably, the vascular smooth muscle cells express vascular smooth muscle cell markers, such as a smooth muscle actin and calponin, and such cells have spindle forms.

A medium used in Step (5′) can be prepared using a medium for animal cell culture as a basal medium. Examples of basal medium include IMDM medium, Medium 199, Eagle's Minimum Essential Medium (EMEM or MEM), aMEM medium, Dulbecco's modified Eagle's Medium (DMEM), Ham's F12 medium, RPMI 1640 medium, Fischer's medium, and a mixture of any thereof. A medium may further contain serum, or it may be a serum-free medium. According to need, a medium can contain, for example, one or more serum alternatives selected from among, for example, albumin, transferrin, knockout serum replacement (KSR) (a serum alternative for FBS when ES cells are cultured), fatty acid, insulin, collagen precursor, trace elements, 2-mercaptoethanol, and 3′-thiol glycerol. A medium can contain one or more substances selected from among lipid, amino acid, L-glutamine, Glutamax (Invitrogen), non-essential amino acid, vitamin, antibiotics, antioxidants, pyruvic acid, buffer, inorganic salts, N2 supplement (Invitrogen), B27 supplement (Invitrogen), GSK-3α/β inhibitor, and a growth factor such as PDGF-BB. An example of a preferable medium is MEM containing 2% FCS and PDGF-BB.

Culture temperature is about 30° C. to 40° C., and preferably about 37° C., although it is not limited thereto. Culture is conducted in atmosphere containing CO₂, and the preferable CO₂ concentration is about 2% to 5%. While the culture duration is not particularly limited, for example, Step (5′) is preferably performed for at least 3 days.

VEGFR2-positive, TRA1-negative, and VE-cadherin-negative cells can be separated from the cells stained with antibodies reacting with VEGFR2, TRA1, and VE-cadherin with the use of a flow cytometer or other means in accordance with a method well known to a person skilled in the art.

### <Screening Method>

The present invention provides a method for screening for a candidate drug that is useful for treatment or prevention of autosomal dominant polycystic kidney disease. With the screening method involving the use of expression levels of the genes shown in Table 1, Table 2, Table 3, and Table 4 as indicators, the agent for treatment or prevention can be identified.

The method for screening for an agent for treatment or prevention of autosomal dominant polycystic kidney disease of the present invention can comprise the following steps:

(A-1) bringing a candidate substance into contact with somatic cells induced to differentiate from iPS cells derived from a patient with autosomal dominant polycystic kidney disease;

(B-1) measuring the expression level of a single gene or two to all genes selected from the group consisting of the genes shown in Table 1 or Table 3; and

(C-1) when the expression level has decreased in comparison with the case in which the candidate substance has not been brought into contact, determining that the candidate substance is an agent for treatment or prevention of autosomal dominant polycystic kidney disease.

Alternatively, the screening method can comprise the following steps:

(A-2) bringing a candidate substance into contact with somatic cells induced to differentiate from iPS cells derived from a patient with autosomal dominant polycystic kidney disease;

(B-2) measuring the expression level of a single gene or two to all genes selected from the group consisting of the genes shown in Table 2 or Table 4; and

(C-2) when the expression level has increased in comparison with the case in which the candidate substance has not been brought into contact, determining that the candidate substance is an agent for treatment or prevention of autosomal dominant polycystic kidney disease.

Examples of somatic cells induced to differentiate from iPS cells include tubular cells, collecting tubule cells, bile duct cells, hepatic cells, pancreatic ductal cells, pancreatic cells, intestinal cells, germ cells, vascular endothelial cells, and vascular smooth muscle cells, with vascular endothelial cells or vascular smooth muscle cells being preferable. Methods for producing tubular cells, collecting tubule cells, bile duct cells, hepatic cells, pancreatic ductal cells, pancreatic cells, intestinal cells, germ cells, vascular endothelial cells, or vascular smooth muscle cells from iPS cells are not particularly limited. These cells can be adequately extracted from the embryoid body or the developed teratoma (e.g., JP 2006-239169 A). Hepatic cells can be produced by the methods disclosed in WO 2006/082890, JP 2010-75631 A, or Hay D C, et al., Proc. Natl. Acad. Sci., U.S.A., 105, 12301-6, 2008, although the methods are not particularly limited thereto. Also, pancreatic cells can be produced by the method disclosed in WO 2007/103282. iPS cells, vascular endothelial cells, or vascular smooth muscle cells can be produced by the method described above.

A method for screening for an agent for treatment or prevention of autosomal dominant polycystic kidney disease preferably involves the use of vascular endothelial cells and such method can comprise the following steps:

(A-3) bringing a candidate substance into contact with vascular endothelial cells induced to differentiate from iPS cells derived from a patient with autosomal dominant polycystic kidney disease;

(B-3) measuring the expression level of a single gene or two to all genes selected from the group consisting of the genes shown in Table 1; and

(C-3) when the expression level has decreased in comparison with the case in which the candidate substance has not been brought into contact, determining that the candidate substance is an agent for treatment or prevention of autosomal dominant polycystic kidney disease.

Alternatively, a screening method can comprise the following steps:

(A-4) bringing a candidate substance into contact with vascular endothelial cells induced to differentiate from iPS cells derived from a patient with autosomal dominant polycystic kidney disease;

(B-4) measuring the expression level of a single gene or two to all genes selected from the group consisting of the genes shown in Table 2; and

(C-4) when the expression level has increased in comparison with the case in which the candidate substance has not been brought into contact, determining that the candidate substance is an agent for treatment or prevention of autosomal dominant polycystic kidney disease.

A method for screening for an agent for treatment or prevention of autosomal dominant polycystic kidney disease preferably involves the use of vascular smooth muscle cells and such method can comprise the following steps:

(A-5) bringing a candidate substance into contact with vascular smooth muscle cells induced to differentiate from iPS cells derived from a patient with autosomal dominant polycystic kidney disease;

(B-5) measuring the expression level of a single gene or two to all genes selected from the group consisting of the genes shown in Table 3; and

(C-5) when the expression level has decreased in comparison with the case in which the candidate substance has not been brought into contact, determining that the candidate substance is an agent for treatment or prevention of autosomal dominant polycystic kidney disease.

Alternatively, a screening method can comprise the following steps:

(A-6) bringing a candidate substance into contact with vascular smooth muscle cells induced to differentiate from iPS cells derived from a patient with autosomal dominant polycystic kidney disease;

(B-6) measuring the expression level of a single gene or two to all genes selected from the group consisting of the genes shown in Table 4; and

(C-6) when the expression level has increased in comparison with the case in which the candidate substance has not been brought into contact, determining that the candidate substance is an agent for treatment or prevention of autosomal dominant polycystic kidney disease.

In the present invention, the expression level of the gene may be detected with the use of the disease marker. According to another embodiment, detection may be carried out with the use of a reporter gene regulated by the transcription regulatory region of the gene.

In the present invention, the transcription regulatory regions of the genes shown in Table 1, Table 2, Table 3, and Table 4 can be isolated from the genome library on the basis of the nucleotide sequence information of the genes of interest. A cell containing a reporter gene regulated by a transcription regulatory region of the gene of interest can be prepared by introducing a vector comprising a reporter gene sequence operably linked to the sequence of the transcription regulatory region into a cell. Alternatively, a reporter gene sequence may be inserted to be operably linked to a site downstream of the transcription regulatory region via homologous recombination by a method well known to a person skilled in the art.

The vector introduction and homologous recombination described above may be carried out in any case in somatic cells, iPS cells, vascular endothelial cells, or vascular smooth muscle cells. Homologous recombination is preferably carried out in iPS cells.

In the present invention, an adequate reporter gene well known in the art can be used. Examples thereof include, but are not particularly limited to, a green fluorescent protein (GFP), a yellow fluorescent protein (YFP), a red fluorescent protein (RFP), luciferase, β glucuronidase (GUS), β-galactosidase, HRP, and chlorum phenycol acetyl transferase.

In the screening method of the present invention, any candidate substance can be used. Examples thereof include, but are not limited to, a cell extract, a cell culture supernatant, a microbial fermentation product, a marine organism extract, a plant extract, a purified or crude protein, a peptide, a nonpeptide compound, a synthetic low-molecular-weight compound, and a natural compound.

In the present invention, a candidate substance can also be obtained by any means selected from among many combinatorial library techniques known in the art including: (1) biological library technique; (2) synthetic library technique employing deconvolution; (3) one-bead one-compound library technique; and (4) synthetic library technique employing affinity chromatography selection. While the biological library technique involving affinity chromatography selection is limited to a technique using a peptide library, the other four techniques are applicable to techniques using peptide, nonpeptide oligomer, or low-molecular-weight compound libraries (Lam, 1997, Anticancer Drug, Des. 12: 145-67). Examples of molecular library synthesis techniques can be found in the art (DeWitt et al., 1993, Proc. Natl. Acad. Sci. U.S.A., 90: 6909-13; Erb et al., 1994, Proc. Natl. Acad. Sci. U.S.A., 91: 11422-6; Zuckermann et al., 1994, J. Med. Chem. 37: 2678-85; Cho et al., 1993, Science 261: 1303-5; Carell et al., 1994, Angew. Chem. Int. Ed. Engl. 33: 2059; Carell et al., 1994, Angew. Chem. Int. Ed. Engl. 33: 2061; Gallop et al., 1994, J. Med. Chem. 37: 1233-51). Compound library can be prepared in the form of solution (see Houghten, 1992, Bio/Techniques 13: 412-21), bead (Lam, 1991, Nature 354: 82-4), chip (Fodor, 1993, Nature 364: 555-6), bacteria (U.S. Pat. No. 5,223,409), spore (U.S. Pat. Nos. 5,571,698, 5,403,484, and 5,223,409), plasmid library (Cull et al., 1992, Proc. Natl. Acad. Sci. U.S.A., 89: 1865-9), or phage (Scott and Smith, 1990, Science 249: 386-90; Devlin, 1990, Science 249: 404-6; Cwirla et al., 1990, Proc. Natl. Acad. Sci. U.S.A., 87: 6378-82; Felici, 1991, J. Mol. Biol. 222: 301-10; US Patent No. 2002103360).

### EXAMPLES

The present invention is described in greater detail with reference to the following examples, although the technical scope of the present invention is not limited to these examples.

### Example 1

### Fibroblasts

The skin samples obtained via biopsy from 7 patients with autosomal dominant polycystic kidney disease, with the consent of such patients, were cultured, and the resultants were used as PK fibroblasts. Separately, dermal fibroblast samples obtained from 7 Japanese individuals who had not developed autosomal dominant polycystic kidney disease were used as nonPK fibroblasts.

### <iPS Cell Induction>

Human cDNAs of Oct3/4, Sox2, Klf4, and c-Myc were introduced into the fibroblasts with the use of the retrovirus in accordance with the method described in Takahashi, K. et al., Cell, 131 (5), 861, 2007. Similarly, human cDNAs of Oct3/4, Sox2, and Klf4 were introduced into the fibroblasts with the use of the retrovirus in accordance with the method described in Nakagawa, M. et al., Nat. Biotechnol., 26 (1), 101, 2008. The fibroblasts were transferred onto SNL feeder cells 6 days after gene introduction, and the medium was exchanged with a primate ES cell culture medium supplemented with 4 ng/ml bFGF (Wako) on the following day. The developed colonies were picked, a single type of iPS cell strain was selected for each fibroblast, and 7 types of PK fibroblast-derived iPS cell strains (PK-iPSC) and 7 types of nonPK fibroblast-derived iPS cell strains (nonPK-iPSC) were prepared.

### Example 2

### Induction of Differentiation into Vascular Endothelial Cells

iPS cell colonies were broken into segments of adequate size, dispersed on a type I collagen-coated dish (IWAKI), and cultured in a primate ES/iPS cell culture medium (ReproCELL) for 1 day, so as to allow the cell colonies to adhere to the dish surface. GSK-3α/β inhibitor (Sigma), N2 supplement, and B27 supplement (Invitrogen) were added on the following day, and culture was conducted for an additional 3 days. The medium was exchanged with a serum-free medium for human hematopoietic stem cell culture (Invitrogen), 50 ng/ml VEGF (Peprotec Inc.) was added, culture was conducted for an additional 5 days, the cells were detached, and VEGFR2-positive, TRA1-60-negative, and VE-cadherin-positive cells were separated via FACS. Subsequently, the separated cells were dispersed in a type IV collagen-coated dish (Becton Dickinson) and cultured in a vascular endothelial cell growth medium (Lonza). When a vascular endothelial cell sheet expressing vascular endothelial cell markers, such as VE-cadherin, CD31, CD34, and eNOS, and exhibiting a cobblestone appearance was constructed, the cells were recovered as vascular endothelial cells (EC). ECs were prepared from 7 types of PK-iPSC and 7 types of nonPK-iPSC (PK-EC and nonPK-EC).

### Example 3

### Induction of Differentiation into Vascular Smooth Muscle Cells

iPS cell colonies were broken into pieces of adequate sizes, dispersed on a type I collagen-coated dish (IWAKI), and cultured in a primate ES/iPS cell culture medium (ReproCELL) for 1 day, so as to allow the cell colonies to adhere to the dish surface. GSK-3α/β inhibitor (Sigma), N2 supplement, and B27 supplement (Invitrogen) were added on the following day, and culture was conducted for an additional 3 days. The medium was exchanged with a serum-free medium for human hematopoietic stem cell culture (Invitrogen), culture was conducted for an additional 5 days, the cells were detached, and VEGFR2-positive, TRA1-60-negative, and VE-cadherin-negative cells were separated via FACS. Subsequently, the separated cells were dispersed in a type I collagen-coated dish (IWAKI) and further cultured in MEM containing 2% FCS and 20 ng/ml PDGF-BB (Peprotec Inc.). The cultured cells were induced to differentiate into vascular smooth muscle cells (SMC) expressing vascular smooth muscle cell markers, such as a smooth muscle actin and calponin, and exhibiting spindle forms, and the resulting cells were recovered. SMCs (PK-SMC and nonPK-SMC) were prepared from the 7 types of PK-iPSC and 7 types of nonPK-iPSC.

### Example 4

### Confirmation of Gene Expression

RNAs extracted from PK-EC and nonPK-EC were applied to the microarrays (Agilent Technologies), so as to identify the genes exhibiting significant differences in expression by 2 times or more. Table 1 shows the genes exhibiting expression levels 2 times higher in PK-EC and Table 2 shows the genes exhibiting expression levels 2 times lower in PK-EC.

TABLE 1

| Gene | Accession No. |
|---|---|
| IGFBP7 | NM_001253835 |
| | NM_001553 |
| IGF1 | NM_00 0618 |
| | NM_001111283 |
| | NM_001111284 |
| | NM_001111285 |
| CPE | NM_001873 |
| CNPY4 | NM_152755 |
| VTN | NM_000638 |
| PCSK1 | NM_000439 |
| | NM_001177875 |
| OLFML2A | NM_001282715 |
| | NM_182487 |
| NPTX2 | NM_002523 |
| LAMC3 | NM_006059 |
| IGFBP3 | NM_000598 |
| | NM_001013398 |
| HTRA1 | NM_002775 |
| GPC4 | NM_001448 |
| CPXM2 | NM_198148 |
| COL5A1 | NM_000093 |
| | NM_001278074 |
| COL15A1 | NM_001855 |
| CLEC4M | NM_001144904 |
| | NM_001144905 |
| | NM_001144906 |
| | NM_001144907 |
| | NM_001144908 |
| | NM_001144909 |
| | NM_001144910 |
| | NM_001144911 |
| | NM_014257 |
| AMH | NM_000479 |
| EEF1A1 | NM_001402 |
| STAG2 | NM_001042749 |
| | NM_001042750 |
| | NM_001042751 |
| | NM_001282418 |
| | NM_006603 |
| SLN | NM_003063 |
| ZSCAN1 | NM_182572 |
| ZNF135 | NM_001164527 |
| | NM_001164529 |
| | NM_001164530 |
| | NM_001289401 |
| | NM_001289402 |
| | NM_003436 |
| | NM_007134 |
| ZDHHC9 | NM_001008222 |
| | NM_016032 |
| TPCN1 | NM_001143819 |
| | NM_001301214 |
| | NM_017901 |
| TNIK | NM_001161560 |
| | NM_001161561 |
| | NM_001161562 |
| | NM_001161563 |
| | NM_001161564 |
| | NM_001161565 |
| | NM_001161566 |
| | NM_015028 |
| TNFSF4 | NM_001297562 |
| | NM_003326 |
| TMEM63C | NM_020431 |
| SULT4A1 | NM_014351 |
| ST6GALNAC1 | NM_001289107 |
| | NM_018414 |
| SRPX2 | NM_014467 |
| SPOCK1 | NM_004598 |
| SNX10 | NM_001199835 |
| | NM_001199837 |
| | NM_001199838 |
| | NM_013322 |
| SLC20A2 | NM_001257180 |
| | NM_001257181 |
| | NM_006749 |
| SEZ6L2 | NM_001114099 |
| | NM_001114100 |
| | NM_001243332 |
| | NM_001243333 |
| | NM_012410 |
| | NM_201575 |
| SELE | NM_000450 |
| RSPO4 | NM_001029871 |
| | NM_001040007 |
| RSPO3 | NM_032784 |
| RGS11 | NM_001286485 |
| | NM_001286486 |
| | NM_003834 |
| | NM_183337 |
| RGCC | NM_014059 |
| RAMP1 | NM_005855 |
| RAI2 | NM_001172732 |
| | NM_001172739 |
| | NM_001172743 |
| | NM_021785 |
| RAB11FIP1 | NM_001002814 |
| | NM_025151 |
| PSORS1C1 | NM_014068 |
| NKAIN4 | NM_152864 |
| MSL3 | NM_001193270 |
| | NM_001282174 |
| | NM_006800 |
| | NM_078628 |
| | NM_078629 |
| LOX | NM_001178102 |
| | NM_002317 |
| KIF1A | NM_001244008 |
| | NM_004321 |
| HSD17B6 | NM_003725 |
| GRIN2D | NM_000836 |
| GLIPR2 | NM_001287010 |
| | NM_001287011 |
| | NM_001287012 |
| | NM_001287013 |
| | NM_001287014 |
| | NM_022343 |
| FZD10 | NM_007197 |
| FBLN5 | NM_006329 |
| CRABP1 | NM_004378 |
| COL1A2 | NM_000089 |
| CD209 | NM_001144893 |
| | NM_001144894 |
| | NM_001144895 |
| | NM_001144896 |
| | NM_001144897 |
| | NM_001144899 |
| | NM_021155 |
| C1S | NM_001734 |
| | NM_201442 |
| BDNF | NM_001143805 |
| | NM_001143806 |
| | NM_001143807 |
| | NM_001143808 |
| | NM_001143809 |
| | NM_001143810 |
| | NM_001143811 |
| | NM_001143812 |
| | NM_001143813 |
| | NM_001143814 |
| | NM_001143816 |
| | NM_001709 |
| | NM_170731 |
| | NM_170732 |
| | NM_170733 |
| | NM_170734 |
| | NM_170735 |

TABLE 2

| Gene | Accession No. |
|---|---|
| PRSS36 | NM_001258290 |
| | NM_001258291 |
| | NM_173502 |
| CMA1 | NM_001836 |
| HERC2 | NM_004667 |
| HAPLN2 | NM_021817 |
| TOR3A | NM_022371 |
| CHAT | NM_001142929 |
| | NM_001142933 |
| | NM_001142934 |
| | NM_020549 |
| | NM_020984 |
| | NM_020985 |
| | NM_020986 |
| COL11A2 | NM_001163771 |
| | NM_080679 |
| | NM_080680 |
| | NM_080681 |
| DPEP3 | NM_001129758 |
| | NM_022357 |
| MDGA1 | NM_153487 |
| OR10A5 | NM_178168 |
| S100A5 | NM_002962 |
| SFTPA2 | NM_001098668 |
| APOC1 | NM_001645 |
| APOL1 | NM_001136540 |
| | NM_001136541 |
| | NM_003661 |
| | NM_145343 |
| CD14 | NM_000591 |
| | NM_001040021 |
| | NM_001174104 |
| | NM_001174105 |
| HNRNPA3 | NM_194247 |
| TAPP | NM_000415 |
| LYNX1 | NM_023946 |
| | NM_177457 |
| | NM_177458 |
| | NM_177476 |
| | NM_177477 |
| MMP9 | NM_004994 |
| NETO1 | NM_001201465 |
| | NM_138966 |
| | NM_138999 |
| NPB | NM_148896 |
| OXT | NM_000915 |
| PHGDH | NM_006623 |
| SLC6A17 | NM_001010898 |
| ARHGEF10 | NM_014629 |
| COX7A1 | NM_001864 |
| FAM57B | NM_031478 |
| LRRD1 | NM_001161528 |
| MYO3A | NM_017433 |
| POT1 | NM_001042594 |
| | NM_015450 |
| CLEC12B | NM_001129998 |
| | NM_205852 |
| DNAH17 | NM_173628 |
| FAM24B | NM_001204364 |
| | NM_152644 |
| HIST1H2AG | NM_021064 |
| HIST1H3J | NM_003535 |
| HOPX | NM_001145459 |
| | NM_001145460 |
| | NM_032495 |
| | NM_139211 |
| | NM_139212 |
| IL1RL1 | NM_001282408 |
| | NM_003856 |
| | NM_016232 |
| KCNC3 | NM_004977 |
| KCNK17 | NM_001135111 |
| | NM_031460 |
| KCTD19 | NM_001100915 |
| KIAA1257 | NM_020741 |
| LOC101929959 | XM_011518093 |
| | XM_006716901 |
| | XM_011518094 |
| MAB21L2 | NM_006439 |
| MED29 | NM_017592 |
| MIR124-2HG | NR_034102 |
| | NR_034103 |
| | NR_109792 |
| | NR_109793 |
| NUTM2D | NR_075100 |
| SCN3A | NM_001081676 |
| | NM_001081677 |
| | NM_006922 |
| SNORA16B | NR_004389 |
| TSPYL5 | NM_033512 |
| WDR90 | NM_145294 |
| YPEL4 | NM_145008 |

Similarly, RNAs extracted from PK-SMC and nonPK-SMC were applied to the microarrays (Agilent Technologies), so as to identify the genes exhibiting significant differences in expression level (i.e., by 2 times or more). Table 3 shows the genes exhibiting expression levels 2 times higher in PK-SMC and Table 4 shows the genes exhibiting expression levels 2 times lower in PK-SMC.

TABLE 3

| Gene | Accession No. |
|---|---|
| ADAMTSL4 | NM_001288607 |
| | NM_001288608 |
| | NM_019032 |
| | NM_025008 |
| COL9A3 | NM_001853 |
| EMILIN2 | NM_032048 |
| CNPY4 | NM_152755 |
| C1QL4 | NM_001008223 |
| EGFL8 | NM_030652 |
| HSPG2 | NM_001291860 |
| | NM_005529 |
| SLITRK4 | NM_001184749 |
| | NM_001184750 |
| | NM_173078 |
| EEF1A1 | NM_001402 |
| PLAC9 | NM_001012973 |
| SLIT2 | NM_001289135 |
| | NM_001289136 |
| | NM_004787 |
| SPANXC | NM_022661 |
| SUSD2 | NM_019601 |
| TMEM255A | NM_001104544 |
| | NM_001104545 |
| | NM_017938 |
| TMEM97 | NM_014573 |
| SLN | NM_003063 |
| STAG2 | NM_001042749 |
| | NM_001042750 |
| | NM_001042751 |
| | NM_001282418 |
| | NM_006603 |
| APPL1 | NM_012096 |
| CALB2 | NM_001740 |
| | NM_007088 |
| CDT1 | NM_030928 |
| CLK1 | NM_001162407 |
| | NM_004071 |
| COLEC12 | NM_130386 |
| DLG2 | NM_001142699 |
| | NM_001142700 |
| | NM_001142702 |
| | NM_001206769 |
| | NM_001300983 |
| | NM_001364 |
| DRD2 | NM_000795 |
| | NM_016574 |
| ENTPD8 | NM_001033113 |
| | NM_198585 |
| FOXB1 | NM_012182 |
| ITGB1BP2 | NM_001303277 |
| | NM_012278 |
| KAT2A | NM_021078 |
| L3MBTL1 | NM_015478 |
| | NM_032107 |
| NUF2 | NM_031423 |
| | NM_145697 |
| QTRT1 | NM_031209 |
| SAPCD1 | NM_001039651 |
| SCARA3 | NM_016240 |
| | NM_182826 |
| SLC8A2 | NM_015063 |
| SNORD31 | NR_002560 |
| SUSD5 | NM_015551 |
| TGM1 | NM_000359 |
| TNNT1 | NM_001126132 |
| | NM_001126133 |
| | NM_001291774 |
| | NM_003283 |
| ZFP42 | NM_001304358 |
| | NM_174900 |
| ZNRF3 | NM_001206998 |
| | NM_032173 |

TABLE 4

| Gene | Accession No. |
|---|---|
| HAPLN2 | NM_021817 |
| TOR3A | NM_022371 |
| WNT10B | NM_003394 |
| PRSS36 | NM_001258290 |
| | NM_001258291 |
| | NM_173502 |
| CHAT | NM_001142929 |
| | NM_001142933 |
| | NM_001142934 |
| | NM_020549 |
| | NM_020984 |
| | NM_020985 |
| | NM_020986 |
| COL11A2 | NM_001163771 |
| | NM_080679 |
| | NM_080680 |
| | NM_080681 |
| DPEP3 | NM_001129758 |
| | NM_022357 |
| MDGA1 | NM_153487 |
| OR10A5 | NM_178168 |
| AHSA2 | NM_152392 |
| CHMP1A | NM_001083314 |
| | NM_002768 |
| EPS8L3 | NM_024526 |
| | NM_133181 |
| | NM_139053 |
| GDF7 | NM_182828 |
| GPC6 | NM_005708 |
| MARK2 | NM_001039469 |
| | NM_001163296 |
| | NM_001163297 |
| | NM_004954 |
| | NM_017490 |
| MROH7 | NM_001039464 |
| | NM_001291332 |
| PLOD1 | NM_000302 |
| S100A5 | NM_002962 |
| SFTPA2 | NM_001098668 |
| ARHGEF10 | NM_014629 |
| COX7A1 | NM_001864 |
| FAM57B | NM_031478 |
| LRRD1 | NM_001161528 |
| MYO3A | NM_017433 |
| POT1 | NM_001042594 |
| | NM_015450 |
| ADAMTS7 | NM_014272 |
| AKT2 | NM_001243027 |
| | NM_001243028 |
| | NM_001626 |
| CBX3 | NM_007276 |
| | NM_016587 |
| CCDC33 | NM_001287181 |
| | NM_025055 |
| | NM_182791 |
| CSAG1 | NM_001102576 |
| | NM_153478 |
| CSAG2 | XM_006724857 |
| CSAG3 | NM_001129826 |
| | NM_001129828 |
| CXCL16 | NM_001100812 |
| | NM_022059 |
| DKK3 | NM_001018057 |
| | NM_013253 |
| | NM_015881 |
| ETV6 | NM_001987 |
| GP9 | NM_000174 |
| GREM1 | NM_001191322 |
| | NM_001191323 |
| | NM_013372 |
| GRIN3A | NM_133445 |
| HLA-DRB5 | NM_002125 |
| IL33 | NM_001199640 |
| | NM_001199641 |
| | NM_033439 |
| KLHL29 | NM_052920 |
| LOC284379 | NR_002938 |
| MAGEA2 | NM_001282501 |
| | NM_001282502 |
| | NM_001282504 |
| | NM_001282505 |
| | NM_005361 |
| | NM_175742 |
| | NM_175743 |
| MAGEA2B | NM_153488 |
| OPN4 | NM_001030015 |
| | NM_033282 |
| PLAU | NM_001145031 |
| | NM_002658 |
| POLR2H | NM_001278698 |
| | NM_001278699 |
| | NM_001278700 |
| | NM_001278714 |
| | NM_001278715 |
| | NM_006232 |
| PRB3 | NM_006249 |
| PRPH2 | NM_000322 |
| PYGO2 | NM_138300 |
| SNORA34 | NR_002968 |
| SSX2 | NM_001278697 |
| | NM_003147 |
| | NM_175698 |
| SSX2B | NM_001164417 |
| | NM_001278701 |
| | NM_001278702 |
| ST14 | NM_021978 |
| SUSD4 | NM_001037175 |
| | NM_017982 |
| TBXAS1 | NM_001061 |
| | NM_001130966 |
| | NM_001166253 |
| | NM_001166254 |
| | NM_030984 |
| TFPI2 | NM_001271003 |
| | NM_001271004 |
| | NM_006528 |
| UBE3B | NM_001270449 |
| | NM_001270450 |
| | NM_001270451 |
| | NM_130466 |
| | NM_183415 |
| WDR1 | NM_005112 |
| | NM_017491 |
| WDR93 | NM_001284395 |
| | NM_001284396 |
| | NM_020212 |
| XKR9 | NM_001011720 |
| | NM_001287258 |
| | NM_001287259 |
| | NM_001287260 |

When the expression levels of the genes shown in Table 1 are higher in ECs derived from the iPS cells prepared from the subject than the levels in the control, it is highly likely that the subject is afflicted with autosomal dominant polycystic kidney disease, on the basis of the results demonstrated above. When the expression levels of the genes shown in Table 2 are higher in ECs derived from the iPS cells prepared from the subject than the levels in the control, in contrast, it is highly likely that the subject is not afflicted with autosomal dominant polycystic kidney disease.

When the expression levels of the genes shown in Table 3 are higher in SMCs derived from the iPS cells prepared from the subject than the levels in the control, it is highly likely that the subject is afflicted with autosomal dominant polycystic kidney disease. When the expression levels of the genes shown in Table 4 are higher in SMCs derived from the iPS cells prepared from the subject than the levels in the control, in contrast, it is highly likely that the subject is not afflicted with autosomal dominant polycystic kidney disease.

### INDUSTRIAL APPLICABILITY

The present invention provides a method for testing for autosomal dominant polycystic kidney disease and a method for screening for an agent for treatment of such disease. Accordingly, the present invention is very useful in the medical field.

1. A method for determining whether or not a subject has developed or is at risk of developing autosomal dominant polycystic kidney disease comprising the following steps:
(a) measuring the expression level of a single gene or two to all genes selected from the group consisting of the genes shown in Table 1 or Table 3 in a sample obtained from the subject; and
(b) when the expression level is higher than the expression level of the same gene in a control sample, determining that the subject has developed or is at risk of developing autosomal dominant polycystic kidney disease.

2. A method for determining whether or not a subject has developed or is at risk of developing autosomal dominant polycystic kidney disease comprising the following steps:
(a) measuring the expression level of a single gene or two to all genes selected from the group consisting of the genes shown in Table 2 or Table 4 in a sample obtained from the subject; and
(b) when the expression level is higher than the expression level of the same gene in a control sample, determining that the subject has not developed or is not at risk of developing autosomal dominant polycystic kidney disease.

3. The method according to claim 1, wherein the sample obtained from the subject is at least one type of sample selected from the group consisting of blood, serum, plasma, cell extract, urine, lymph, tissue fluid, ascites fluid, spinal fluid, another body fluid, a tissue, and a cell.

4. The method according to claim 1, wherein the sample obtained from the subject is a vascular endothelial cell induced to differentiate from the iPS cell derived from a somatic cell of the subject and the gene in Step (a) is selected from the group consisting of the genes shown in Table 1.

5. The method according to claim 2, wherein the sample obtained from the subject is a vascular endothelial cell induced to differentiate from the iPS cell derived from a somatic cell of the subject and the gene in Step (a) is selected from the group consisting of the genes shown in Table 2.

6. The method according to claim 1, wherein the sample obtained from the subject is a vascular smooth muscle cell induced to differentiate from the iPS cell derived from a somatic cell of the subject and the gene in Step (a) is selected from the group consisting of the genes shown in Table 3.

7. The method according to claim 2, wherein the sample obtained from the subject is a vascular smooth muscle cell induced to differentiate from the iPS cell derived from a somatic cell of the subject and the gene in Step (a) is selected from the group consisting of the genes shown in Table 4.

8. A method for screening for an agent for treatment or prevention of autosomal dominant polycystic kidney disease comprising the following steps:
(a) bringing a candidate substance into contact with a vascular endothelial cell induced to differentiate from the iPS cell derived from a somatic cell of a patient with autosomal dominant polycystic kidney disease;
(b) measuring the expression level or transcription activity of a single gene or two to all genes selected from the group consisting of the genes shown in Table 1 and Table 2; and
(c) when the expression level or transcription activity of a single gene or two to all genes selected from the group consisting of the genes shown in Table 1 has decreased in comparison with the case in which the candidate substance has not been brought into contact, determining that the candidate substance is an agent for treatment or prevention of autosomal dominant polycystic kidney disease, or when the expression level or transcription activity of a single gene or two to all genes selected from the group consisting of the genes shown in Table 2 has increased, selecting the candidate substance as an agent for treatment or prevention of autosomal dominant polycystic kidney disease.

9. A method for screening for an agent for treatment or prevention of autosomal dominant polycystic kidney disease comprising the following steps:
(a) bringing a candidate substance into contact with a vascular smooth muscle cell induced to differentiate from the iPS cell derived from a somatic cell of a patient with autosomal dominant polycystic kidney disease;
(b) measuring the expression level or transcription activity of a single gene or two to all genes selected from the group consisting of the genes shown in Table 3 and Table 4; and
(c) when the expression level or transcription activity of a single gene or two to all genes selected from the group consisting of the genes shown in Table 3 has decreased in comparison with the case in which the candidate substance has not been brought into contact, determining that the candidate substance is an agent for treatment or prevention of autosomal dominant polycystic kidney disease, or when the expression level or transcription activity of a single gene or two to all genes selected from the group consisting of the genes shown in Table 4 has increased, selecting the candidate substance as an agent for treatment or prevention of autosomal dominant polycystic kidney disease.

10. The screening method according to claim 8, wherein the step of measuring the gene expression level comprises measuring the mRNA, cRNA, or cDNA level of the gene.

11. The method according to claim 2, wherein the sample obtained from the subject is at least one type of sample selected from the group consisting of blood, serum, plasma, cell extract, urine, lymph, tissue fluid, ascites fluid, spinal fluid, another body fluid, a tissue, and a cell.

12. The screening method according to claim 9, wherein the step of measuring the gene expression level comprises measuring the mRNA, cRNA, or cDNA level of the gene.
